(19) United States
(12) Patent Application Publication
(10) Pub. No.: US 2005/0260190 A1
(43) Pub. Date: Nov. 24, 2005

(54) Therapeutic agent for iNOS generating illness

(76) Inventors: Robert J. Webber, Benicia, CA (US); Douglas S. Webber, Benicia, CA (US); Thelma H. Dixon, Benicia, CA (US)

Correspondence Address: THEODORE J. BIELEN JR., 1390 WILLOW PASS ROAD, SUITE 1020, CONCORD, CA, 94520, US

(21) Appl. No.: 10/849,768

(22) Filed: May 19, 2004

(51) Int. Cl. A61K 39/395

(52) U.S. Cl. 424/130.1

(57) ABSTRACT

A therapeutic agent which removes or neutralizes iNOS in the blood of a mammalian subject. The agent may be in the form of an anti-iNOS monoclonal antibody or an INOS binding entity.

### BACKGROUND OF THE INVENTION

The present invention relates to a novel and useful therapeutic agent for the removal or neutralization of particulate iNOS in the blood, providing treatment for systemic inflammatory response syndrome (pre-sepsis), sepsis, severe sepsis, and septic shock.

Nitric oxide synthase (NOS) is an enzyme which is found in humans. Three isoforms of NOS have been identified. In the body nNOS and eNOS are constitutively expressed in the cells in which they are found. However, iNOS is not constitutively expressed, but is known to be induced by a number of cytokines lypopolysaccarides (LPS), and other mediators of the inflammatory response. Specifically, iNOS has been associated as indicating certain pathological disease states. Notably, iNOS in the blood heralds the onset of sepsis, severe sepsis, and septic shock conditions in humans. Sepsis is estimated to kill more than 200,000 people annually in the United States alone. Of the persons who develop sepsis thirty percent die from this pathophysiology.

Reference is made to U.S. Pat. No. 6,531,578 in which monoclonal antibodies are described that are specific for the recognition of iNOS in humans without cross-reacting with human eNOS or nNOS. U.S. Pat. No. 6,531,578 is incorporated by reference in whole to the present application. An immunoassay using such monoclonal antibody is capable of detecting the presence of sepsis within a very short period of time, a matter of minutes, when compared to the prior art tests which required several days to complete and obtain results. If sepsis is treated aggressively after recognition of its existence, persons afflicted with sepsis have a much better chance of surviving. Treatment of sepsis has been limited to known antibacterial, antifungal, and antiviral treatments. Such treatments have achieved limited success even with the rapid recognition of the presence of sepsis in a human.

An article entitled “Cloning and Characterization of Inducible Nitric Oxide Synthase from Mouse Macrophages”, Xie et al, Science, 256: 225-228 (1992), reported the cloning and isolation of iNOS. The iNOS enzyme was described as a soluble cytoplasmic protein.

Subsequently, articles entitled “Nitric Oxide: Novel Biology with Clinical Relevance”, Billiar, Ann Surg, 221#4: 339-349 (1995); “Nitric Oxide: Pathophysiological Mechanisms”, Gross et al, Annu Rev Physiol, 57: 737-769 (1995); “The Cell Wall Components Peptidoglycan and Lipoteichoic Acid from Staphylococcus Aureus Act in Synergy to Cause Shock and Multiple Organ Failure”, De Kimpe et al, Proc Natl Acad Sci USA, 92: 10359-10363 (1995); “Mechanism of Gram-Positive Shock: Identification of Peptidoglycan and Lipoteichoic Acid Moieties Essential in the Induction of Nitric Oxide Synthase, Shock, and Multiple Organ Failure”, Kengatharan et al, J Exp Med, 188#2: 305-315 (1998); and “Induction of Nitric Oxide Synthase in RAW 264.7 Macrophages by Lipoteichoic Acid from Staphylococcus Aureus: Involvement of Protein Kinase C- and Nuclear Factor-κB-Dependent Mechanisms”, Kuo et al, J Biomed Sci, 10: 136-145 (2003), point to the fact that the lipopolysaccharide (LPS) cell-wall component of gram-negative bacteria, the lipoteichoic acid and peptidoglycan cell-wall components of gram-positive bacteria, fungi, and viruses can induce INOS expression in vivo and in vitro in a wide variety of cell types.

Articles entitled “Mechanisms Of Suppression Of Macrophage Nitric Oxide Release By Transforming Growth Factor Beta”, Vodovotz et al, J Exp Med, 178#2: 605-613 (1993); “Vesicle Membrane Association Of Nitric Oxide Synthase In Primary Mouse Macrophages”, Vodovotz et al, J Immunol, 154#6: 2914-2925 (1995); and “Bladder Instillation And Intraperitoneal Injection Of Escherichia Coli Lipopolysaccharide Up-Regulate Cytokines And iNOS In Rat Urinary Bladder”, Olsson et al, J Pharmacol Exp Ther, 284#3: 1203-1208 (1998), have shown that since the discovery of iNOS in mouse macrophages, its intracellular location is not exclusively in the cytosol. In fact vesicle-associated iNOS has been recognized.

Articles entitled “Caveolin-1 Down-Regulates Inducible Nitric Oxide Synthase Via The Proteasome Pathway In Human Colon Carcinoma Cells”, Felley-Bosco E et al, Proc Natl Acad Sci USA, 97#26: 14334-14339 (2000); “Macrophage Nitric Oxide Synthase Associates With Cortical Actin But Is Not Recruited To Phagosomes”, Infect Immun, Webb J L et al, 69#10: 6391-6400 (2001); “Epithelial Inducible Nitric-Oxide Synthase Is An Apical EBP50-Binding Protein That Directs Vectorial Nitric Oxide Output”, Glynne P A et al, J Biol Chem, 277#36: 33132-33138 (2002); “Caveolin-1-Mediated Post-Transcriptional Regulation Of Inducible Nitric Oxide Synthase In Human Colon Carcinoma Cells”, Felley-Bosco E, Biol Res, 35#2: 169-176 (2002); “Heat Shock Protein 90 As An Endogenous Protein Enhancer Of Inducible Nitric-Oxide Synthase”, Yoshide M et al, J Biol Chem, 278#38: 36953-36958 (2003); “Protein Interactions With Nitric Oxide Synthase: Controlling The Right Time, The Right Place, And The Right Amount Of Nitric Oxide”, Kone B C et al, Am J Physiol Renal Physiol, 285#2: F178-F190 (2003); and “Protein-Protein Interactions Involving Inducible Nitric Oxide Synthase”, Zhang W et al, Acta Physiol Scand, 179#2: 137-142 (1997), have also reported that when induced cells are lysed and fractionated by centrifugation, iNOS is found in the particulate fraction.

Inducible NOS (iNOS) has also been found associated with a number of other proteins through a protein-protein interaction. Such protein-protein interactions (other proteins associated with iNOS) include cortical actin, EBP 50 (ezrin-redixin-moesin-binding phosphoprotein 50), caviolin-1, Hsp90 (heat shock protein 90), kalirin, NAP110 (NOS-associated protein 1.10 kd), and Rac-GTPases. These protein-protein interactions have been found to localize iNOS to specific regions or structures within cells. Upon cell lysis and fractionation by centrifugation, either through vesicle association or by protein-protein interaction, a portion of the supposedly soluble iNOS protein has been shown to partition into the particulate fraction.

U.S. patent application Ser. No. 09/628,585, revealed the fact that iNOS found free in the liquid portion of the blood of a patient, i.e. plasma, indicates such patient has sepsis or will develop sepsis within the next 24 to 48 hours. Using a very sensitive chemiluminescent sandwich enzyme immunoassay (EIA), such plasma iNOS can be used as a very specific biochemical marker for the onset of sepsis. The heretofore referenced chemiluminescent sandwich (EIA) was based upon two of the anti-iNOS monoclonal antibodies (MAbs) of a panel of anti-iNOS antibodies which are disclosed in U.S. Pat. No. 6,531,578, mentioned heretofore.

Although the detection of iNOS in the blood of patients is greatly aided in the treatment of those patients by conventional therapies, an improved therapy would be a notable advance in the medical field.

### BRIEF SUMMARY OF THE INVENTION

In accordance with the present invention a novel and useful therapeutic agent for an illness generating iNOS in the blood of a patient is herein provided.

The therapeutic agent of the present invention may take the form of a monoclonal antibody recognizing human iNOS without cross-reacting with eNOS or nNOS. In this regard, the iNOS recognized is believed to comprise the particulate fraction of the iNOS. Such monoclonal antibody may neutralize particulate iNOS which may be found in membrane-associated particulate iNOS, vesicle-associated particulate iNOS, or particulate iNOS in association with at least one other protein. It has been found that the illness most normally associated with the generation of iNOS in the blood of a patient is systemic inflammatory response syndrome (pre-sepsis), sepsis, severe sepsis, or septic shock. Moreover, the monoclonal antibody may be mouse anti-iNOS monoclonal antibody, mouse-human chimeric anti-iNOS monoclonal antibody, humanized anti-iNOS monoclonal antibody, or human anti-iNOS monoclonal antibody. Also, the therapeutic treatment of the present invention is capable of removing iNOS from the blood of a mammalian subject by association with the monoclonal antibody. In such a case, means for achieving this result is also provided in the present invention. Such means may take the form of a device coated with a monoclonal antibody which binds human iNOS without cross-reacting with eNOS or nNOS.

Instead of an anti-iNOS monoclonal antibody being used as a therapeutic agent, an iNOS binding entity may also be employed. For example, iNOS binding aptmers, oligionucleotides, artificial antibodies, phage displayed antibodies, phage displayed antibody fragments, and single-chain monoclonal antibodies may be used in this regard. Such therapeutic agents have been animal tested and are believed to serve as positive treatments for maladies or illness inducing iNOS in the blood of the patient.

It may be apparent that a novel and useful therapeutic agent for the treatment of an illness in a mammalian subject generating iNOS has been hereinabove described.

It is therefore an object of the present invention to provide a therapeutic agent for the treatment of an illness in a subject generating iNOS which is safe and effective.

Another object of the present invention is to provide a therapeutic agent for the treatment of a malady in a mammalian subject generating iNOS which either neutralizes or removes iNOS from the blood of the patient.

Another object of the present invention is to provide a therapeutic agent for the treatment of a malady in a mammalian subject generating iNOS which is easily manufactured using known biochemical and immunological techniques.

A further object of the present invention is to provide a therapeutic agent for the treatment of an illness in a mammalian subject generating INOS in the blood which is capable of saving lives.

The invention possesses other objects and advantages especially as concerns particular characteristics and features thereof which will become apparent as the specification continues.

### BRIEF DESCRIPTION OF THE SEVERAL VIEWS OF THE DRAWING

FIG. 1 is a photograph of a field of peripheral blood mononuclear cells (PBMCS) from a patient showing only one immunostaining positive cell (white), located at arrowhead, and a small iNOS containing vesicle (white), located at arrow, reacting with the indirect-fluorescent-labeled anti-iNOS monoclonal antibody 2A1-F8 in a field with numerous other non-reacting cells (very pale), at 200×.

FIG. 2 is a photograph of a field of PBMCS showing a large percentage of the cells containing iNOS and cellular associated iNOS containing vesicles (arrows) which are immunostained with the anti-iNOS monoclonal antibody 2A1-F8 by an indirect immunofluorescent assay (IFA) procedure, at 200×.

FIG. 3 is a photograph showing a less crowded field of PBMCS from that of FIG. 2 from a different septic patient than FIG. 2, in which iNOS-containing vesicle (presumably apoptotic bodies) are separate from the cells, at 200×.

FIG. 4 is a photograph having three panels, A, B, and C in which a common area is shown sequentially in UV light(A), phase-contrast light(B), and a combination of UV and phase-contrast light(C), at 200×, showing a large cluster of iNOS-containing vesicles (arrows).

FIG. 5 is a photograph of a PBMC from a patient photographed in UV light indicating a partially disrupted cytoplasmic membrane associated with iNOS-containing globules (presumably pre-apoptotic bodies), revealed by the IFA reaction with anti-iNOS monoclonal antibody 2A1-F8, at 400×.

FIG. 6 is a photograph of a PBMC photographed in UV light from the same patient as that shown in FIG. 5 indicating an iNOS immunopositive staining cell in the process of disintegration, and releasing iNOS-containing vesicles (presumably apoptotic bodies) at 200×.

FIG. 7 is a photograph of a western immunoblot showing the soluble and particulate fractions of iNOS where, lane 1 is molecular weight standards, lane 2 is the induced soluble fraction at 5 μl, lane 3 is the induced soluble fraction at 2.5 μl, lane 4 is the induced particulate fraction at 5 μl, lane 5 is the induced particulate fraction at 2.5 μl, lane 6 is an iNOS standard, and lane 7 is the molecular weight standards.

FIG. 8 is a chart illustrating the 48 hour survival of mice primed with LPS and four hours later administered the chemical entities described in Example 1.

FIG. 9 is a western immunoblot following SDS-PAGE indicating the removal of iNOS from the particulate fraction described in Example 2, in which lane 1 is the molecular weight standards, lane 2 is an iNOS standard at 5 μl, lane 3 is the induced particulate fraction at 5 μl, lane 4 is the induced particulate fraction at 2.5 μl, lane 5 is the anti-iNOS MAb coated MAG-BEAD depleted particulate fraction at 5 μl, lane 6 is the anti-iNOS MAb coated MAG-BEAD depleted particulate fraction at 2.5 μl, lane 7 is an INOS standard at 5 μl, and lane 8 is the molecular weight standards.

FIG. 10 is a photograph of the immunostaining of iNOS bound to anti-iNOS monoclonal antibodies attached to the MAG-BEADs used in Example 2.

FIG. 11 is a chart illustrating the seven day survival of mice primed with LPS and four hours later administered certain chemical entities, described in Example 2.

FIG. 12 is a chart illustrating the seven day survival of mice primed with LPS and four hours later administered certain chemical entities, described in Example 3.

FIG. 13 is a chart illustrating the seven day survival of mice primed with LPS and four hours later administered certain chemical entities, described in Example 4.

For a better understanding of the invention reference is made to the following detailed description of the preferred embodiments thereof which should be taken in conjunction with the prior described drawings.

### DETAILED DESCRIPTION OF THE PREFERRED EMBODIMENTS OF THE INVENTION

Various aspects of the present invention will evolve from the following detailed description of the preferred embodiments thereof and examples which should be taken together with the hereinbefore described drawings.

In clinical trials, more than 340 human subjects were enrolled, and over 1,200 blood samples were collected and analyzed to determine if the chemilumenescent EIA for iNOS, described in U.S. Pat. No. 6,531,578 and U.S. patent application Ser. No. 09/628,585 prognosticate the onset of sepsis and monitored the course of the pathology. It was found that free iNOS (soluble iNOS) was present in the blood samples. Also, particulate iNOS, in the form of membrane associated particulate iNOS, vesicle associated particulate iNOS, or particulate iNOS in association with another protein (by protein-protein interaction), was present in some of the blood samples. Such particulate iNOS was not part of any cells.

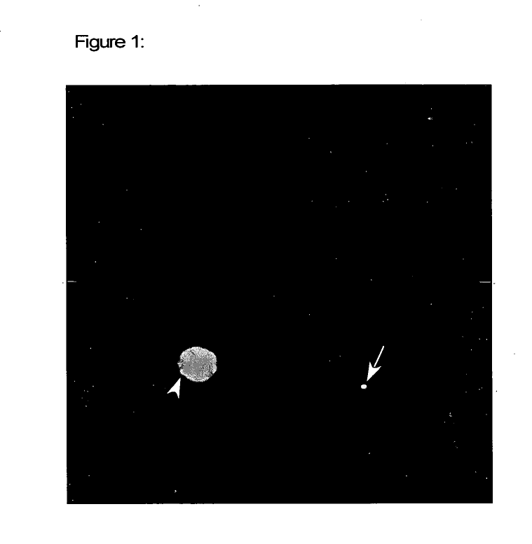

FIGS. 1-6 showed the presence of human iNOS in peripheral blood mononuclear cells (PBMCs) and in vesicles/globules that are not cell associated. For example, FIG. 1 shows a PBMC cell that contains human iNOS. The iNOS-containing PBMC cell is present in a background of non-staining cells. It may be observed that the immunostaining in this PBMC cell is not evenly distributed, as might be expected by the typical distribution of cytoplasmic protein in a normal cell. The immunostaining material appears globular and is located at the peripheral rim of the cytoplasm (arrow head). The nuclear region does not contain iNOS and appears pale as a result of the thin layers of cytoplasm above and below the nuclear in the cell. Also, a single vesicle appears, that is not cell associated, which also contains human iNOS (full arrow). It is believed that the single vesicle which is intensely fluorescing may be an apoptotic body.

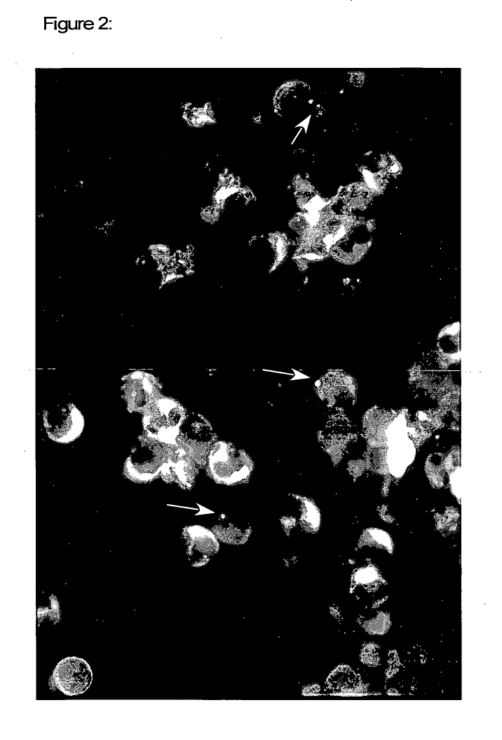

Turning to FIG. 2, many cells are shown each of which are immunostaining for human iNOS. Also visible are numerous small extra cellular vesicles (apoptotic bodies), that are immunostaining for human iNOS, too. The presumptively apoptotic bodies are present as small white dots located at the edges of the cells (full arrows). The nucleus of each cell is present in the non-fluorescently staining portions (dark region) of the cells. Also, other non-reacting cells are present in the background.

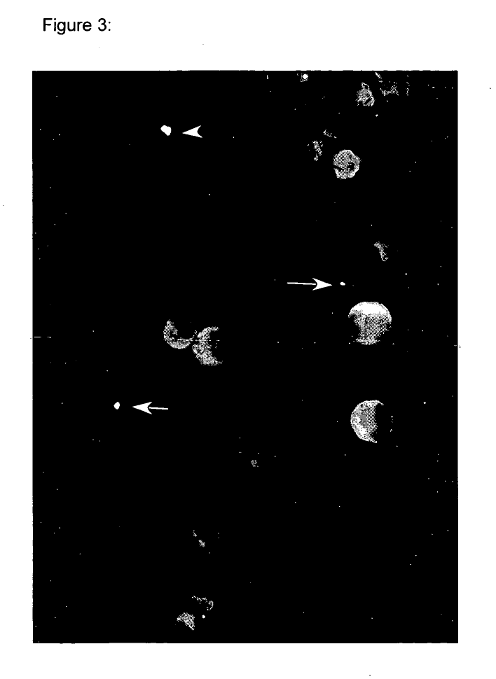

FIG. 3 depicts a relatively open field of PBMCS. Small extra cellular vesicles, presumably apoptotic bodies, appear as white dots and are separate from the cells (arrows). The immunostaining for iNOS with the anti-iNOS monoclonal antibody 2A1-F8 appears granular in these cells. The anti-iNOS monoclonal antibody 2A1-F8 is disclosed in U.S. Pat. No. 6,531,578.

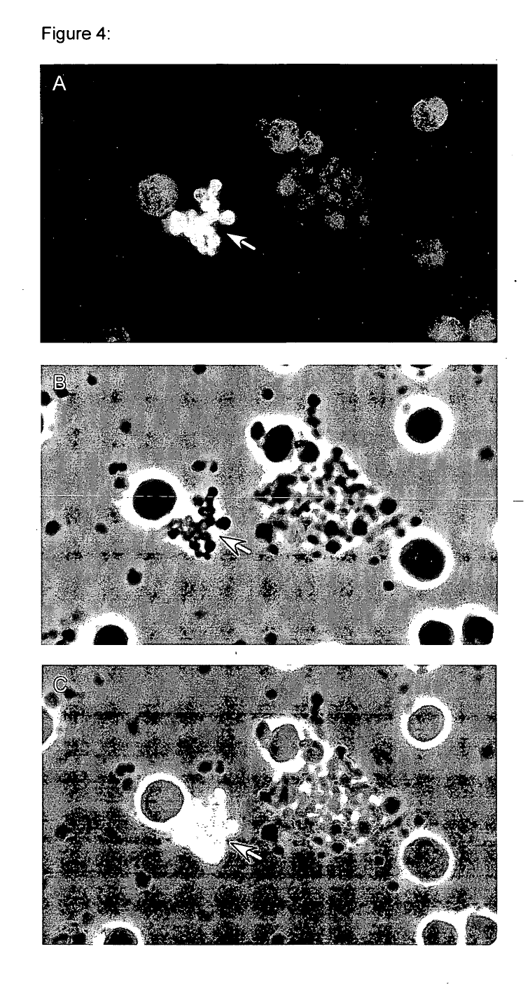

FIG. 4 depicts three photographs, in panels A, B, and C, with a common cluster of globules and cells. Panel A was photographed in UV light and reveals the immunostaining of globules located as a cluster. Some of the cells appear small, are shrunken, and are negative for INOS immunostaining by IFA. An arrow appears next to the immunostained globules in a cluster.

Panel B, FIG. 4, was photographed in phase-contrast light, and again reveals the presence of the numerous extra cellular globules in the sample (arrow). The intact cells are shown as dark black bodies with a white halo that the results from the phase-contrast optics.

Finally, in panel C of FIG. 4, the same area as shown in panels A and B was photographed with a combination of UV light and phase-contrast optics. The white cluster of globules (arrow) indicate immunostaining with anti-iNOS monoclonal antibody 2A1-F8, demonstrating that the extra-cellular globules contain iNOS.

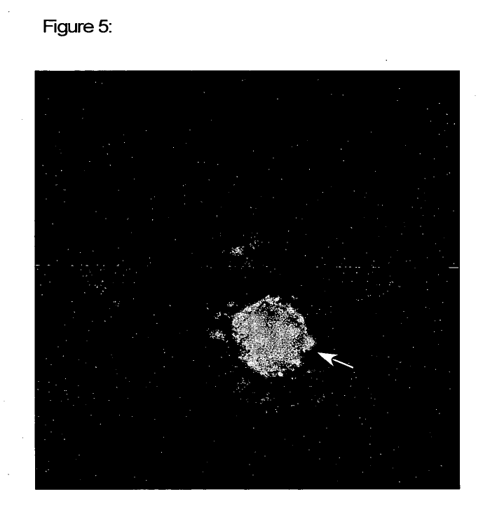

With reference to FIG. 5, a single PBMC, immunostaining for human iNOS, is in the process of “blebbing”. In other words, the PBMC cell has a partially disrupted cytoplasmic membrane associated with iNOS containing globules. The pre-apoptotic bodies or apoptotic “blebs” are immunostained for human iNOS.

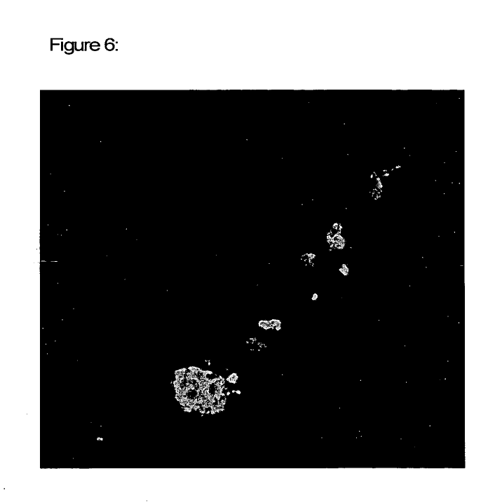

FIG. 6 depicts a single PBMC cell in the process of disintegration and releasing materials and vesicles that immunostained for human iNOS. The cell membrane has been disrupted and the iNOS-containing globules/vesicles are scattered.

FIGS. 1-6 represent evidence for the existence of apoptotic bodies in vivo. All of the particulate or vesicle-associated iNOS was only found in samples from patients afflicted with sepsis, severe sepsis, or septic shock. The presence of apoptotic bodies as revealed in FIGS. 1-6 in the blood stream of a human may be an indication of the presence of sepsis or an indication of the severity of the pathology of the same.

Since soluble iNOS and the particulate or vesicle-associated iNOS are only found in the blood of critically ill patients, the contribution to the pathology of systemic inflammatory response syndrome, sepsis, severe sepsis, or septic shock by these forms of iNOS was investigated. In other words, the presence of soluble or particulate iNOS in the circulation was theorized to be deleterious to patients with sepsis or pre-sepsis. Consequently, it may be reasoned that removal or neutralization of iNOS from the blood stream of patients with sepsis or pre-sepsis conditions may constitute a possible therapeutic treatment for such illnesses.

In order to gather data that might confirm such hypothesis, a mouse model of sepsis was used in a series of experiments. Such tests were employed to determine whether or not soluble or particulate iNOS contributed to the pathology of systemic inflammatory response syndrome sepsis, severe sepsis, or septic shock. In addition, it was determined if removal or the neutralization of soluble or particulate iNOS helps improve the sepsis pathology.

As heretofore stated, DLD-1-5B2 cells can be induced to produce human INOS by the addition of a mixture of cytocytokines.

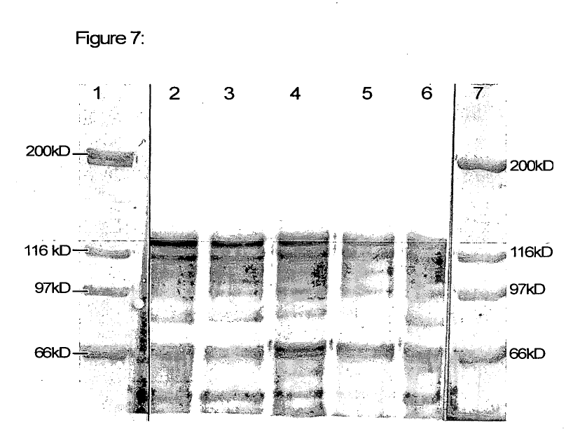

In an article entitled “Transcriptional Regulation of Human Inducible Nitric Oxide Synthase Gene In An Intestinal Epithelial Cell Line”, Linn et al, Am J Physiol, 272: G1499-G1508 (1997), it was shown that DLD-1-5B2 cells can be induced to produce human iNOS. Also, when the induced cells are lysed, two types of iNOS can be isolated by centrifugation, a soluble iNOS fraction and a particulate iNOS fraction. FIG. 7 shows a Western immunoblot which indicates that the pooled soluble fraction of induced DLD-1-5B2 cells contains iNOS (lanes 2 and 3) at the predicted molecular weight of 131 kD. Also, the particulate fraction of induced DLD-1-5B2 cells likewise contains iNOS (lanes 4 and 5) as shown by the band at 131 kD. Lane 6 contains an iNOS standard and lanes 1 and 7 contain standard proteins used as molecular weight markers at the indicated weights. In order to produce and isolate soluble and particulate fractions of iNOS from induced DLD-1-5B2 cell cultures, the following steps were followed:
1. DLD-1-5B2 cells were grown in culture starting from frozen cryo-preserved cells;
2. The expression of iNOS was induced in the cells;
3. The induced cells were harvested; and
4. The iNOS in the induced cells was isolated and fractionated into soluble and particulate fractions.

To grow and expand DLD-1-5B2 cells, a vile of cryo-preserved cells was obtained and thawed. The percent viability was calculated—it should be greater than 75 percent—by trypan blue exclusion prior to culturing the cells. Cells were transferred into a T-75 flask containing DLD-1-5B2 medium (90 percent DMEM and 10 percent FBS supplemented with PEN/Strept). Cells were incubated in a humidified atmosphere of 5 percent CO₂ in air at 37° C. The medium was changed every other day until the cells were almost confluent. Following such procedure, the medium was changed daily until the cell cultures were either split or induced. When the DLD-1-5B2 cells were near confluence in log-phase growth, the cells were split 1:6 to 1:10 into additional T-75 flasks.

DLD-1-5B2 cells were induced to express human iNOS using a mixture of rhIFNγ at 8.33 ng/ml, rhTNFa at 3.3 ng/ml, and rhI-L-1 at 3.3 ng/ml for 18 hours. During the induction of iNOS, an increase in the quantity of the end products of NO production, nitrite and nitrate, was monitored to determine if iNOS was being produced by the induced cells. The Griess reaction was used to assay the quantity of nitrite contained in the culture medium, and the amount of nitrate after the enzymatic conversion of the nitrate to nitrite by the enzyme nitrate reductase.

The DLD-1-5B2 cells were harvested, 18 hours post-induction. To maximize induce cell recovery, all culture fluid from the induced flasks were transferred and combined into 50 ml sterile centrifuge tubes to collect “floater” cells. Each tube was centrifuged, and the spent medium was discarded. The “floater” cell pellet was set aside until ready to wash with PBS. All the T-75 flasks were washed with PBS to remove spent medium and its serum components. A mixture of trypsin/EDTA was incubated for five to ten minutes at 37° C. to dislodge the cells from the surface of each flask. After the cells were freed from the plastic surface, heat-inactivated FBS was added to each flask to stop the trypsin reaction. The cells were then transferred to a screw capped centrifuge tube and collected by centrifugation. “Floater” cells were then combined with the trypsinized cell pellet. The pooled induced cells were washed three times with sterile PBS and collected by centrifugation after each wash. The washed cells were transferred into sterile tubes in a small volume of sterile PBS, and stored at −20° C. until ready to process for the isolation and fractionation of iNOS.

The iNOS produced by the induced DLD-1-5B2 cells was then fractionated into soluble and a particulate fractions. The induced DLD-1-5B2 cells, which were previously harvested and frozen, were thawed in an ice water bath until the entire contents of the tube had melted. Occasional vortexing during the thawing aided the process. The cells were lysed by two rapid freeze/thaw cycles using dry ice. The lysed cells were centrifuged at 16,000 xg at 4° C. for 30 minutes to pellet the particulate fraction. The supernatant containing the soluble iNOS was transferred and stored on ice. The pellets were resuspended in a small volume of ice cold sterile PBS, vortex mixed vigorously, and centrifuged at 16,000×g at 4° C. for 30 minutes to pellet the particulate fraction. The resulting supernatant was pooled with the first supernatant. Such supernatant solution contained the isolated INOS soluble fraction. The particulate fraction of INOS was then stored at −20° C. or used. The iNOS soluble fraction required stabilization by the addition to a final concentration of 2 percent normal horse serum, followed by storage in a frozen condition at −20° C.

In general, once the cryo-pressured cells are restarted, they reach log phase growth after a few days of culturing at 37° C. in a humidified 5 percent CO₂/95 percent air atmosphere. Once the DLD-1-5B2 cells are in log-phase growth, a daily monitoring and feeding cycle is required for maximum iNOS expression, the DLD-1-5B2 cells should be induced with a mixture of three cytokines (IFNγ, TNFα, and IL-β) two days past confluence and harvested 18 hours after the start of the induction. From starting up the cell culture to finishing the first harvest took approximately 11 days, inductions and harvests were a weekly routine thereafter. DLD-1 cells are available from ATCC (CAT.#CCL221). The DLD-1-5B2 cell lines may be derived by subcloning the DLD-1 cells using standard cloning techniques.

The effects of the soluble iNOS fraction and of the particulate iNOS fraction were tested on mice primed with a sub-lethal dose of LPS as a model of sepsis. This was done to determine the effects the two different fractions of iNOS protein had on the viability of the mice. The results of these experiments led to the discovery that the iNOS protein has a function in signaling death in the mice. Several experiments were conducted, and it was discovered that the membrane associated iNOS, rather than soluble fraction of iNOS, plays a role in causing death in this mouse model of sepsis. It was also discovered that antibodies found in U.S. Pat. No. 6,531,578 protected mice from death caused by a sub-lethal dose of lipopolysaccharide (LPS) and iNOS.

It is also found from the experiments that LPS priming of mice was necessary for the effect of the particulate iNOS to be exerted. Also, the administration of the particulate iNOS fraction, without soluble iNOS, to LPS prime mice caused almost immediate death. Particulate iNOS by itself or in association with one or more proteins is believed to be responsible for the lethal effect observed in LPS prime mice. Removal of the iNOS in particulate form or in association with one or more proteins by absorption from solution stopped the lethal effects exerted by the administration of the particulate INOS to the LPS primed mice. It was also found that different anti-iNOS MAbs varied in their individual ability to neutralize the lethality in the mice of particulate iNOS, in the form of particulate membrane associated iNOS, particulate vesicle associated iNOS or particulate iNOS in association with at least another protein. Also, lowering the dose of LPS and particulate iNOS increased the survival rate of the mice. However, the administration of anti-iNOS MAbs to LPS primed mice increased the seven day survival rate.

The following examples are provided to further illustrate the present invention but are not deemed to limit the invention in any manner.

### EXAMPLE I

The two fractions of human iNOS, illustrated in FIG. 7, were investigated as to their effect on LPS primed mice as an animal model of sepsis. Prior to starting the experiment, soluble iNOS was removed from the soluble fraction by selective absorption onto MAG-BEADS coated with one or more of the anti-iNOS MAbs found in the U.S. Pat. No. 6,531,578. Briefly, MAG-BEADS covalently bonded to goat anti-mouse IgG. IgG were purchased from the Pierce Chemical Co. in Rockford, Ill. Culture supernatant containing secreted anti-iNOS MAbs from clones 21C10-1D10, 2A1-F8, 1E8-B8 and 2D2-B2 were applied individually to aliquots of the suspended MAG-BEADS in order to load the MAG-BEADS with monoclonal antibodies specific for iNOS. The soluble fraction containing iNOS was diluted 1:2 and applied to pooled, washed, a resuspended anti-iNOS coated MAG-BEADS. The suspension was incubated overnight with gentle mixing to allow the soluble iNOS to bind to the anti-iNOS MAbs coated onto the MAG-BEADS before the tube containing the suspension a magnetic was placed onto a magnetic rack. All the beads congregated on the sides of the tube next to the magnets. The resulting iNOS-depleted soluble fraction was transferred and diluted to a final volume to yield a 1:5 dilution as compared to the stock soluble fraction. A 1:5 dilution of the stock iNOS soluble fraction was also prepared in sterile saline. Samples of the 1:5 iNOS-depleted soluble fraction of the 1:5 diluted stock soluble fraction, and of the iNOS coated MAG-BEADS used to create the iNOS-depleted soluble fraction were all analyzed to determine if the soluble iNOS had been removed and to demonstrate that the soluble iNOS was bound to the anti-iNOS MAbs attached to the MAG-BEADS. These analyses showed that more than 90 percent of the soluble iNOS had been removed from the soluble fraction (iNOS-depleted soluble fraction), and that the iNOS was bound to the MAG-BEADS which had been loaded with the anti-iNOS MAbs.

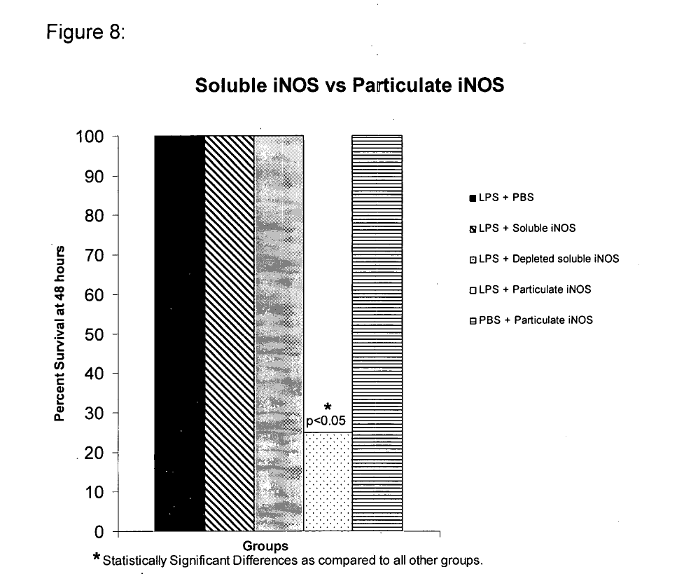

Groups of mice containing both genders were injected IP with sterile saline only, or with a sub-lethal dose of LPS (2 mg/kg body weight of LPS Serotype 0111:B4 from E. coli, obtained from Sigma Chemical Co., Saint Louis, Mo.) in sterile saline. After four hours, only the mice injected with LPS became lethargic and developed diarrhea. The saline or LPS-primed mice were then given an additional tail vein injection of either saline or one of the following: the soluble fraction containing iNOS (soluble iNOS), the soluble fraction depleted of iNOS (iNOS-depleted soluble fraction), or particulate iNOS produced by and isolated from induced DLD-1-5B2 cells. FIG. 8 shows the results of this experiment. None of the saline-primed mice showed any effect with any of the test samples. No effect was seen with the LPS-primed mice upon administration by tail vein injection of either a dose of saline, a dose of soluble iNOS, or a dose of iNOS-depleted soluble fraction. However, when the particulate fraction of iNOS was administered to the LPS prime mice, all the mice died almost immediately. No effect was seen on the saline-primed mice given the same dose of particulate iNOS. It was concluded that (1) LPS priming of mice was necessary for the effect of the particulate iNOS to be exerted, and (2) particulate iNOS, not soluble iNOS, when administered to LPS-primed mice caused an almost immediate death.

### EXAMPLE II

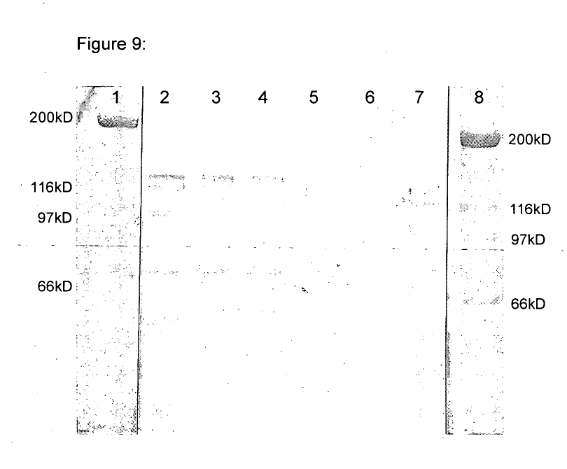

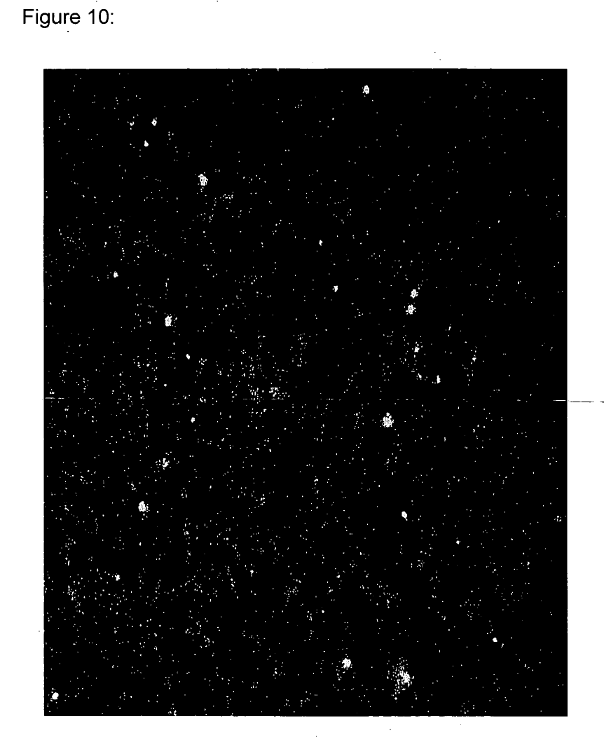

The anti-human iNOS MAbs found in U.S. Pat. No. 6,531,578 were employed in order to investigate the inhibition of the killing effect seen with the particulate human iNOS in LPS-primed mice. Particulate iNOS was removed from the particulate fraction by selective absorption onto MAG-BEADS coated with the anti-iNOS MAbs as described in Example I. FIG. 9 represents the Western immuno blot confirming the selective removal of particulate iNOS from the particulate fraction. A similar procedure to the one described with respect to depletion of the soluble fraction in Example I was employed. Briefly, MAG-BEADS covalently linked to goat anti-mouse IgG. IgG were purchased from the Pierce Chemical Company in Rockford, Ill. Culture supernatants containing secreted anti-iNOS MAbs from clones 21C10-1D10, 2A1-F8, 1E8-B8, and 2D2-B2 were applied individually to aliquots of the suspended MAG-BEADS in order to load the beads with antibodies to iNOS. The particulate fraction containing iNOS was diluted 1:5 and applied to the pooled, wash, and resuspended anti-iNOS coated MAG-BEADS. The suspension was incubated overnight with gentle mixing to allow the particulate iNOS to bind to the antibodies coated to the MAG-BEADS before the tube containing the suspension was placed on the magnetic rack. All the beads congregated to the sides of the tube next to the magnets, and the iNOS-depleted solution (iNOS-depleted particulate fraction) was transferred and diluted to a final volume to yield a 1:10 dilution as compared to the stock particulate fraction. A 1:10 dilution of the stock iNOS particulate fraction was also prepared in sterile saline. Samples of the iNOS-depleted particulate fraction, of the stock particulate fraction, and of the iNOS loaded MAG-BEADS used to create the iNOS-depleted particulate fraction, were all analyzed to determine if the particulate iNOS had been removed, FIG. 9. The iNOS bound to the anti-iNOS MAbs attached to the MAG-BEADS is determined in FIG. 10. These analyses showed that more than 90 percent of the particulate iNOS had been removed from the particulate fraction (iNOS-depleted particulate fraction) and that the iNOS was bound to the MAG-BEADS which had been loaded with the anti-iNOS MAbs.

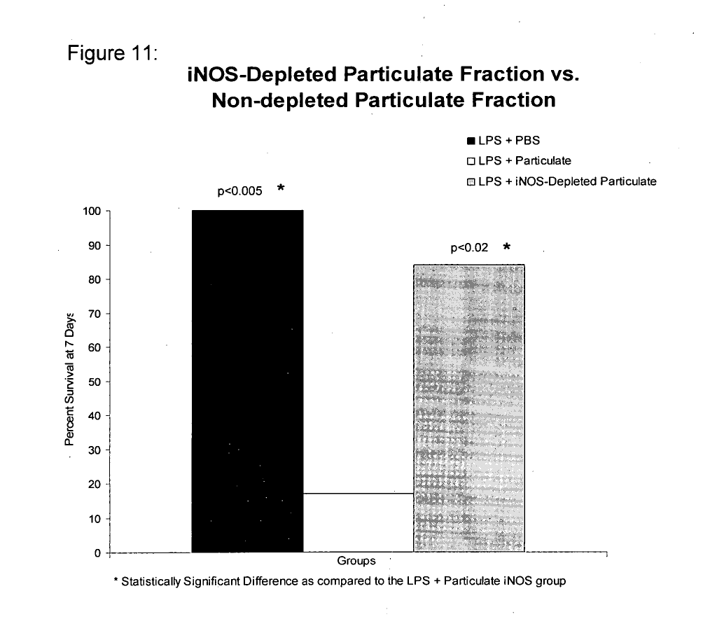

The effect that the iNOS-depleted particulate fraction had on the LPS-primed mice was compared to that seen with the stock (non-depleted) particulate fraction containing particulate iNOS. Groups of mice containing both genders were injected IP with a sub-lethal dose of LPS (2 mg/kg body weight of LPS serotype 0111:B4 from E. coli obtained from the Sigma Chemical Company) in sterile saline. After four hours, all the mice primed with LPS became lethargic and developed diarrhea. The various groups of mice were then given a tail vein injection of either saline, stock particulate iNOS at a 1:10 dilution, or iNOS-depleted particulate fraction at a 1:10 dilution as compared to the staring stock suspension. FIG. 11 represents these definitive results. None of the mice that received a priming IP injection of LPS followed four hours later by a tail injection of saline showed any effect since they all survived seven days until the end of the experiment of this Example. However, only 17 percent (one out of six) of the mice that received a priming IP injection of LPS followed four hours later by a tail injection of particulate iNOS at a 1:10 dilution, survived for seven days. Significantly, 84 percent (five of six) of the LPS-primed mice that received a tail vein injection of the iNOS-deleted particulate fraction survived for seven days. When these data were compared, a high degree of statistically significant difference was found between the survival of the mice administered the particulate iNOS fraction and those administered saline (P<0.005) or the iNOS-depleted particulate fraction (P<0.02). There was no statistically significant difference between the LPS-primed mice that received a saline IV injection and those that received the iNOS-depleted particulate fraction. Thus, the specific removal of the particulate iNOS from the particulate fraction abolished the lethal effect seen in the LPS-primed mice that received the particulate iNOS fraction. It was concluded that (1) LPS priming was required for the lethal effect of particulate iNOS to be exerted; (2) particulate iNOS by itself or particulate iNOS in association with one or more proteins was responsible for the lethal effect observed in LPS-primed mice; and (3) removal of the particulate iNOS or particulate iNOS in association with one or more proteins, by absorption from solution using immobilized anti-iNOS MAbs, stopped the lethal effects asserted by the administration of the particulate iNOS.

### EXAMPLE III

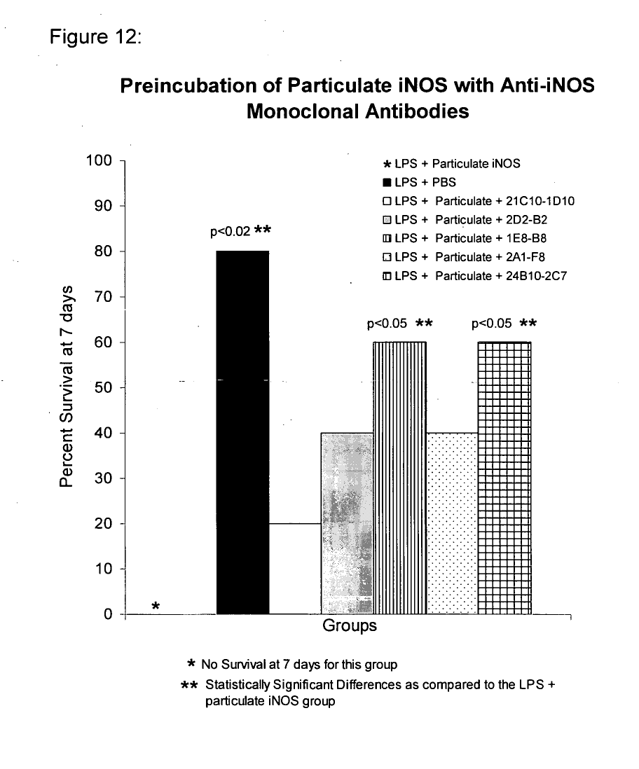

A second method was employed to study the ability of the anti-human iNOS MAbs of U.S. Pat. No. 6,531,578 to inhibit the killing effect seen with particulate human iNOS in LPS-primed mice as a mode for sepsis. Instead of physically removing the particulate iNOS from the particulate fraction as was performed in Example II, individual anti-iNOS MAbs contained in ascites fluid were added directly to aliquots of the particulate fraction that contained particulate iNOS. The particulate iNOS fraction was allowed to bind to the anti-iNOS MAbs for 45 minutes before the material was injected IV into mice. Five different anti-iNOS MAbs were tested for their individual ability to inhibit (neutralize) the killing effect of the particulate human iNOS. Groups of mice were primed with a sub-lethal dose of LPS (2 mg/kg body weight of LPS Serotype 0111:B4 from E. coli obtained from the Sigma Chemical Company) in sterile saline. After four hours all the LPS-primed mice became lethargic and developed diarrhea. The various groups of mice were given a tail vein injection of either saline, stock particulate iNOS at a 1:10 dilution, or stock particulate iNOS at a 1:10 dilution that had been preincubated for 45 minutes with one of five different anti-iNOS MAbs. Each of the five different anti-iNOS MAbs was used at a 1:50 dilution of the ascites fluid. The results varied and are shown in FIG. 12. All the LPS-primed mice that received a tail vein injection of the stock particulate iNOS diluted 1:10 in sterile saline died within the first 24 hours of the seven day experiment. In contrast, four out of five (80 percent) of the LPS-primed mice administered a saline tail vein injection survived seven days (P<0.02). The ability of the anti-iNOS MAbs to neutralize the lethal effect of the particulate iNOS varied depending on the MAb being tested. Of the five different anti-iNOS MAbs tested, anti-iNOS MAb 1E8-B8 and 24B10-2C10 were the best at neutralizing the lethal effects of the particulate iNOS on LPS-primed mice. In both cases, three out of five mice survived seven days (P<0.05). To other anti-iNOS MAbs (2D2-B2 and 2A1-F8) were also somewhat effective in stopping the mice from dying, i.e. two out of five mice in each of these groups survived seven days. One anti-iNOS MAb (21C10-1D10) was much less effective since only one out of five mice survived seven days. It was concluded that: (1) LPS priming is necessary for the particulate iNOS to be lethal; (2) that it is not necessary to remove physically the particulate iNOS from the solution in order to neutralize its lethality; (3) that anti-iNOS MAbs used can neutralize the lethal effects of particulate iNOS on LPS-primed mice by binding to INOS or by binding to the protein-protein complex containing particulate iNOS; and (4) that different anti-iNOS MAbs vary in their individual ability to neutralize the lethality of particulate iNOS either as particulate iNOS itself or in association with one or more proteins.

### EXAMPLE IV

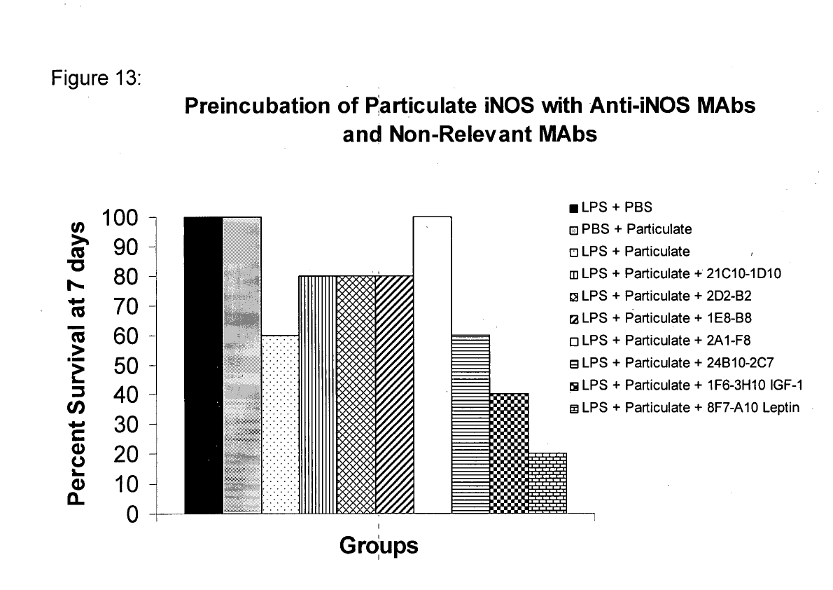

A lower priming dose of LPS and a lower dose of particulate iNOS were employed than that used in Examples I-III on groups of mice. Ascites fluid containing non-relevant MAbs were also tested as controls. The non-relevant controls MAbs included one specific for insulin-like growth factor 1 (IGF-1: MAb clone 1F6-3H10) and one MAb specific for human leptin (MAb clone 8F7-A10). Groups of mice were primed with a lower sub-lethal dose of LPS (1 mg/kg body weight of LPS Serotype 0111:B4 from E. coli obtained from the Sigma Chemical Company) in sterile saline. After four hours, all the LPS primed mice became lethargic and developed diarrhea. The various groups of mice were then give a tail vein injection of either saline, stock particulate iNOS at a 1:20 dilution, or stock particulate iNOS at a 1:20 dilution that had been preincubated for 45 minutes with one of five different anti-iNOS MAbs or one of two non-relevant MAbs, above-identified. The non-relevant MAbs and the anti-iNOS MAbs were each used at a dilution of 1:50 of the ascites fluid. The results were variable and are shown on FIG. 13. By using the lower amount of priming LPS and of particulate iNOS on mice, three out of five mice survived for seven days. As is shown in FIG. 13, anti-iNOS MAb clone 2A1-F8 was best at neutralizing the lethal effects of the particulate iNOS, since five out of five mice survived in this group until the end of the seven day experiment. However, when the particulate iNOS was preincubated with the ascites fluid containing either of the two non-relevant MAbs the survival rate at seven days was lower as compared to the iNOS particulate fraction. This suggests: (1) that one or more components in the ascites fluid increases the lethal effect of the particulate iNOS, or (2) that the individual mice in the group primed with LPS and then administered the particulate iNOS were less sensitive to the lethal effects of the particulate iNOS. Consequently, more members of the group survived than would have been expected to survive under similar conditions. If it is the latter case, then using more mice per group would resolve this statistical problem. If it is the former, where the ascites fluid somehow amplified the lethality of the particulate iNOS, then the ability of the anti-iNOS MAbs to neutralize the lethal effect is being underestimated. When the survival of the group of mice administered the particulate INOS preincubated with anti-iNOS MAb 2A1-FA was compared to the survival of the mice in the two non-related MAb groups, a statistically significant difference was found—in other words, when compared to the group preincubated with an anti-IGF-1 MAb, P<0.05, and when compared to the group preincubated with anti-leptin MAb, P<0.02. It was concluded in this Example that: (1) lower doses of LPS and particulate iNOS increase the seven-day survival rate of the mice; (2) the mice had to be primed with LPS for the lethal effect of the particulate iNOS to be observed; (3) preincubation of the particulate iNOS with anti-iNOS MAbs increase the seven day survival rate of the mice; and (4) ascites fluid by itself was not responsible for the beneficial effects observed with the anti-iNOS MAbs.

While in the foregoing, embodiments and Examples representing the carrying out of the present invention have been set forth in considerable detail for the purposes of making a complete disclosure of the invention, it may be apparent to those of skill in the art that numerous changes may be made in such detail without departing from the spirit and principles of the invention.

1-11. (canceled)

12. A therapeutic agent for the treatment of an illness in a mammalian subject generating iNOS in its blood, comprising:
and iNOS binding entity.

13. The therapeutic agent of claim 12 in which said iNOS binding entity is selected from the group consisting essentially of: iNOS binding aptamers, oligonucleotides, artificial antibodies, phage displayed antibodies, phage displayed antibody fragments, and single-chain monoclonal antibodies.

14. The therapeutic agent of claim 12 in which said iNOS binding entity comprises a particulate iNOS neutralizing binding entity, and the particulate iNOS is selected from the group consisting essentially of: particulate membrane-associated iNOS, particulate vesicle associated iNOS, and particulate iNOS in association with at least another protein.

15. The therapeutic agent of claim 12 which further comprises means for removing the iNOS from the mammalian subject in association with the said iNOS binding entity.

16. The therapeutic agent of claim 15 in which said means for removing iNOS from the mammalian subject in association with said iNOS binding entity includes: a device coated with a binding entity recognizing human iNOS.

17. The therapeutic agent of claim 12 in which the illness is selected from the group consisting essentially of: systemic inflammatory response syndrome, sepsis, severe sepsis, and septic shock.

18. The therapeutic agent of claim 13 which further comprises means for removing the iNOS from the mammalian subject in association with the said iNOS binding entity.

19. The therapeutic agent of claim 18 in which said means for removing iNOS from the mammalian subject in association with said iNOS binding entity includes: a device coated with a binding entity recognizing human iNOS.

20. The therapeutic agent of claim 13 in which said iNOS binding entity comprises a particulate iNOS neutralizing binding entity, and the particulate iNOS is selected from the group consisting essentially of: particulate membrane-associated iNOS, particulate vesicle associated iNOS, and particulate iNOS in association with at least another protein.

21. The therapeutic agent of claim 13 in which the illness is selected from the group consisting essentially of: systemic inflammatory response syndrome, sepsis, severe sepsis, and septic shock.

22. A composition for diagnosing an illness in mammalian subjects generating iNOS in the blood,
comprising:
an immunostained iNOS form.

23. The composition of claim 22 in which said immunostained iNOS form comprises extracellular vesicle associated iNOS.

24. The composition of claim 22 in which the illness comprises systemic inflammatory response syndrome, sepsis, severe sepsis, and septic shock.

25. A method for treating an illness in a mammalian subject generating iNOS in its blood,
comprising the step of:
interacting the blood of the mammalian subject with a monoclonal antibody recognizing human iNOS.

26. A method for treating an illness in a mammalian subject generating iNOS in its blood,
comprising the step of:
interacting the blood of the mammalian subject with an iNOS binding entity.

27. A therapeutic agent for the treatment of an illness generating iNOS in the blood of a mammalian subject,
comprising:
a monoclonal antibody recognizing human iNOS.

28. The agent of claim 27 in which said monoclonal antibody recognizing human iNOS comprises a particulate iNOS neutralizing monoclonal antibody, and the particulate iNOS is selected from the group consisting essentially of: particulate membrane-associated iNOS, particulate vesicle associated iNOS, and particulate iNOS in association with at least another protein.

29. The agent of claim 27 in which the illness is selected form the group consisting essentially of:
systemic inflammatory response syndrome, sepsis, severe sepsis, and septic shock.

30. The agent of claim 27 in which said monoclonal antibody is selected from the group consisting essentially of mouse anti-iNOS monoclonal antibody, mouse-human chimeric anti-iNOS monoclonal antibody, humanized anti-iNOS monoclonal antibody, and human anti-iNOS monoclonal antibody.

31. The agent of claim 27 which further comprises means for removing the iNOS from the mammalian subject in association with said monoclonal antibody recognizing human iNOS.

32. The agent of claim 31 in which the iNOS comprises particulate iNOS and is selected form the group consisting essentially of: particulate membrane associated iNOS, particulate vesicle-associated iNOS, and particulate iNOS in association with at least another protein.

33. The agent of claim 31 in which the illness is selected form the group consisting essentially of: systemic inflammatory response syndrome, sepsis, severe sepsis, and septic shock.

34. The agent of claim 31 in which said monoclonal antibody is selected from the group consisting essentially of mouse anti-iNOS monoclonal antibody, mouse-human chimeric anti-iNOS monoclonal antibody, humanized anti-iNOS monoclonal antibody, and human anti-iNOS monoclonal antibody.

35. The agent of claim 31 in which said means for removing the iNOS from the mammalian subject in association with said monoclonal antibody includes: a device coated with a monoclonal antibody recognizing human iNOS.

36. The agent of claim 35 in which the illness is selected from the group consisting essentially of:
systemic inflammatory response syndrome, sepsis, severe sepsis, and septic shock.

37. The agent of claim 35 in which said monoclonal antibody is selected from the group consisting essentially of: mouse anti-iNOS monoclonal antibody, mouse-human chimeric antibody, and human anti-iNOS monoclonal antibody, humanized anti-iNOS monoclonal antibody, and human anti-iNOS monoclonal antibody.

38. The agent of claim 35 in which said monoclonal antibody recognizing human iNOS comprises a particulate iNOS neutralizing monoclonal antibody, and the particulate iNOS is selected from the group consisting essentially of: particulate membrane-associated iNOS, particulate vesicle associated iNOS, and particulate iNOS in association with at least another protein.
